(19) United States
(12) Patent Application Publication
(10) Pub. No.: US 2005/0274906 A1
(43) Pub. Date: Dec. 15, 2005

(54) Germicidal toothbrush & germicidal toothbrush holder sterilization by directing ultra violet germicidal light by total internal reflection through an optical fiber that conducts light

(76) Inventor: Robert H. Riddell, Bellevue, WA (US)

Correspondence Address: Robert H. Riddell, 4711 116th Ave. South East, Bellevue, WA, 98006, US

(21) Appl. No.: 10/865,877

(22) Filed: Jun. 14, 2004

(51) Int. Cl. A46B 9/04; A61L 2/10

(52) U.S. Cl. 250/455.11; 15/167.1; 15/207.2; 422/24

(57) ABSTRACT

A germicidal toothbrush and cleaner that uses a germicidal light source to disinfect the individual bristles on the toothbrush. The toothbrush includes bristles made of optical fibers capable of transmitting ultraviolet light. There is also a special method of attachment of the toothbrush inside the cleaner that secures the toothbrushes in a set position in the holder. The germicidal light source may be a germicidal fluorescent ultraviolet lamp. The light rays from the germicidal light source are directed at the opposite ends of the bristle at the critical angle, or slightly greater than the critical angle, in order to attain total internal reflection of the light down the bristles of the toothbrushes.

### BACKGROUND OF THE INVENTION

1. Field of the Invention

This invention relates to toothbrushes and devices used to sanitize them.

2. Description of the Related Art

Typically, optical fibers are used in communication cables, which use refractive index, critical angle, and total internal reflection to transmit light signals hundreds of miles. In order to go long distances without a loss of signal, they use coherent infrared light with boosters every so often, and cladding surrounding the core to prevent loss of signal.

The speed of light in the air is approximately 300,000 m/sec, while the speed of light in an optical fiber is slower, approximately 181,000 m/sec. Whenever light passes from one medium to another medium that has a lower speed of light, there will be a change in the direction of light. This change in direction is called the angle of refraction, and it always bends toward the normal. Normal is the line perpendicular to the interface between the air and the optical fiber. See Tables 1 and 2.

There is always a partial reflection of the light up until the critical angle of approximately 43 degrees, after which light is totally reflected. Therefore when light in air meets an optical fiber at an angle of incidence greater than the critical angle, the light will be totally reflected. This is the reason that a light signal can be transmitted for miles down an optical fiber with out much loss of the signal. The diagrams show these angles and how the light passes down an optical fiber.

### SUMMARY OF THE INVENTION

It is an object of the present invention to provide a germicidal toothbrush and germicidal toothbrush cleaner.

It is a further object of this invention to provide a method to sterilize a toothbrush by directing coherent ultraviolet germicidal light, through bristles made of optical fibers that conduct light.

These and other objects are met by the toothbrush disclosed herein that uses bristles made of optical fibers. The bristles are about the same size and position as bristles of a regular toothbrush. The only difference is that they transmit light and function as esthetic and flexible as regular toothbrush bristles.

Also disclosed herein is a toothbrush cleaner which includes a coherent, germicidal, ultraviolet light source to sanitize the bristles and the toothbrush head. The toothbrush cleaner also includes a toothbrush holder designed to hold at least one toothbrush inside the cleaner at an angle relative to the germicidal light source so that the ends of the bristles are positioned at an angle of incidence greater than the critical angle for total internal reflection of the light. Because the bristles are not covered with a cladding, some germicidal light escapes through the sides of the bristles and sanitizes the exterior surfaces of the adjacent bristles, and the surrounding areas on the toothbrush head.

Because the approximate speed of light in the optical fiber can be determined, the critical angle for total internal reflection of the light in the bristle of the toothbrush can easily be set by moving the light source to different positions in the cleaner. One the position of the germicidal light source is determined, further adjustment can be made to the position of the head and bristles by adjusting the spring clamp retainers that hold the heads of the toothbrushes. When all the adjustments are completed, the source of light and the heads of the toothbrushes are locked into position.

### DESCRIPTION OF THE DRAWINGS

FIG. 1 is a top view of a first embodiment of a toothbrush cleaner showing an ultraviolet light source located in a low transversely aligned position in the cleaner's body, with seven toothbrushes located vertically mounted in a door with a reflector also mounted on the door that surrounds the toothbrush heads.

FIG. 2 is a side elevational view of the cleaner shown in FIG. 1.

FIG. 3 is a sectional rear elevational view of the invention shown in FIGS. 1 and 2 showing seven toothbrushes mounted on the door and the light source mounted along the bottom surface.

FIG. 4 is a side elevational view of a second embodiment of the cleaner with the light source mounted and two toothbrushes aligned longitudinally inside the main body with the light source mounted directly across from the toothbrush heads.

FIG. 5 is a right side elevational view of a second embodiment shown in FIG. 4.

FIG. 6 is a top view to showing a toothbrush head being held in the cleaner by a spring clamp retainer.

FIG. 7 is a rear elevational view of the toothbrush mounted in a spring clamp retainer shown in FIG. 6.

### DESCRIPTION OF THE PREFERRED EMBODIMENT(S)

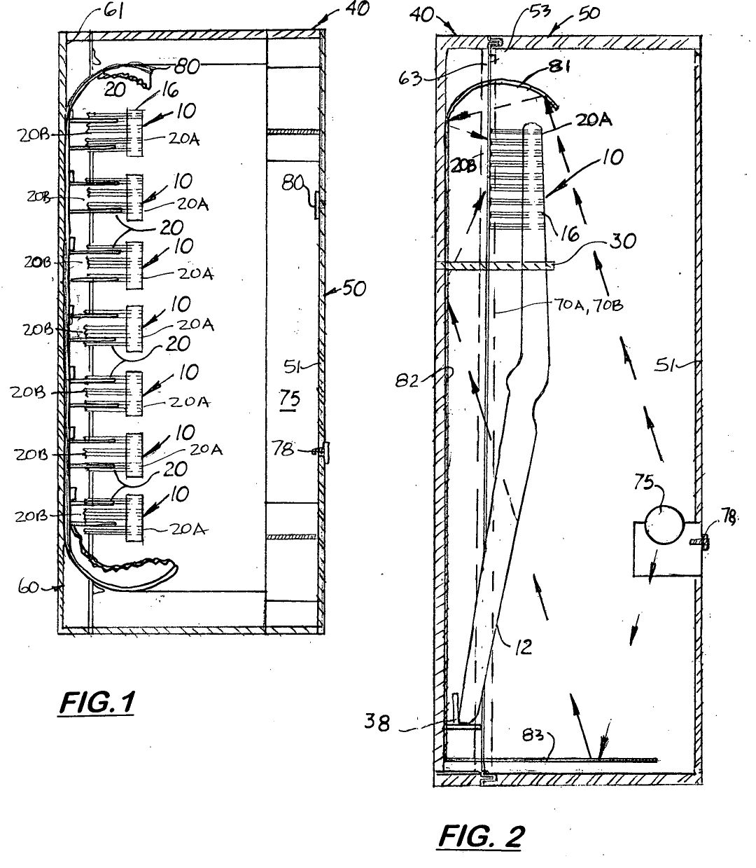

Referring to FIGS. 1-7, there is shown a germicidal toothbrush 10 and a toothbrush cleaner 40 that uses an ultraviolet light source 75 to sanitize the bristles 20 on the toothbrush 10.

The toothbrush 10 includes an elongated handle 12 about the same size and shape as a standard toothbrush with a head 16 located at one end with perpendicularly aligned bristles 20 attached thereto. The bristles 20 extend completely through the head 16 so that their distal and proximal ends 20A, 20B are exposed to light. Each bristle 20 is a single optical fiber capable of transmitting light from one end to the other. Between the end of the handle 12 and the head 16 is a narrow neck area 18, which is engaged by a spring clamp retainer 30 used to securely hold the toothbrush 10 in a set position in the cleaner 40.

In the first embodiment, shown in FIGS. 1-3, the cleaner 40 is an elongated box structure with a rectangular shaped main body 50 and a closable door 60. The main body 50 is approx. 3 inches by 6 inches by 9 inches (W×L×H), and the door 60 is approximately the same size. Hinges 62 are disposed between the adjacent edges of the main body 50 and door 60 that allows the door 60 to be selectively opened and closed. Disposed between the perimeter edges 53, 63 of the main body 50 and the door 60, respectively, are interlocking male and female seals 70A, 70B, respectively, that prevents light from escaping between the adjoining edges 53, 63 of the main body 50 and door 60 when the door 60 is closed. Use of the interlocking seals 70A, 70B is important for preventing light from escaping from the main body 50 and causing injury during use.

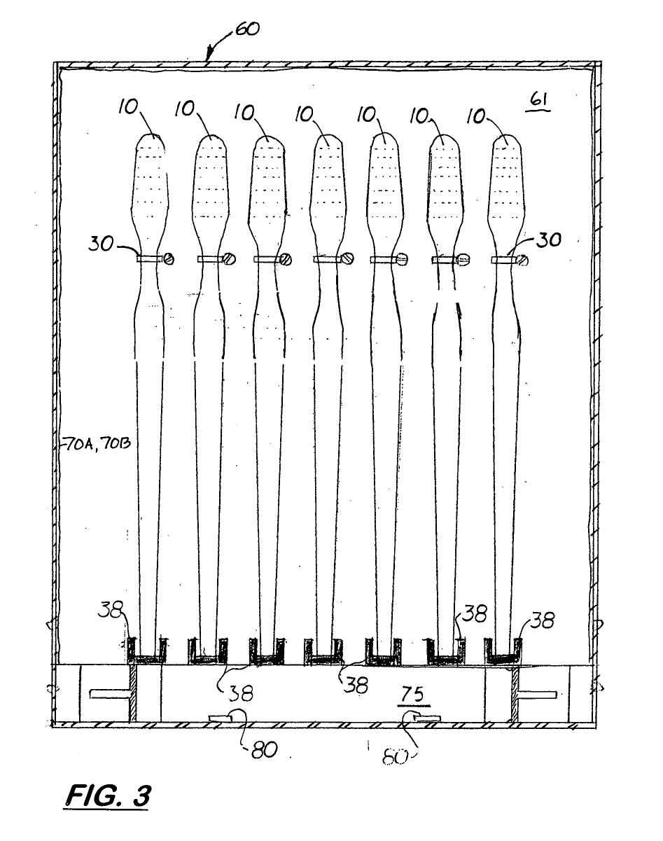

Located inside the cleaner 40 is a germicidal light source 75. In the first cleaner 40, the germicidal light source 75 is an elongated germicidal fluorescent lamp approximately 6 inches in length including the end connectors, and ½ inch in diameter. In the first embodiment shown in FIGS. 1 and 3, the lamp is longitudinally aligned and attached to the back wall 51 of the main body 50 opposite the door 60 with screws 78.

Ultraviolet light is divided into three sections according to wavelength. UV-C includes wavelengths from 100 nm to 280 nm, UV-B includes wavelengths from 280 nm to 315 nm, while UV-A includes wavelengths form 315 nm to 400 nm. Only UV-C light is germicidal which deactivates DNA of bacteria and viruses and so destroys their ability to multiply and cause disease. It specifically damages the nucleic acid of organisms by forming covalent bonds to adjacent bases in the DNA. The formation of such bonds prevents the DNA from unzipping during replication and the organisms is unable to reproduce.

The coherent light from any germicidal lamp rated at 265 nm will have at least 90% of the rays doe to 265 nm.

As discussed below, the light source 75 is designed to be moved to different positions on the back wall 51 by adjusting the screws 78. Typically, the light source 75 is set in a desired position by the manufacturer.

Located on the door 60 and behind the toothbrushes 10 is a reflector 80. The preferred embodiment, the reflector 80 includes an upper concave section 81 that surrounds the toothbrush head, a flat intermediate section 82 that extends downward behind the handle, and a lower section 83 that is perpendicular aligned with the intermediate section 82 that extends forward beyond the end of the handle 12. During operation, the light rays from the germicidal light source 75 is transmitted directly to the proximal ends 20B of the toothbrushes 10 or reflected off the various sections 81-83 of the reflector 80 and towards the distal ends 20A of the bristles 20.

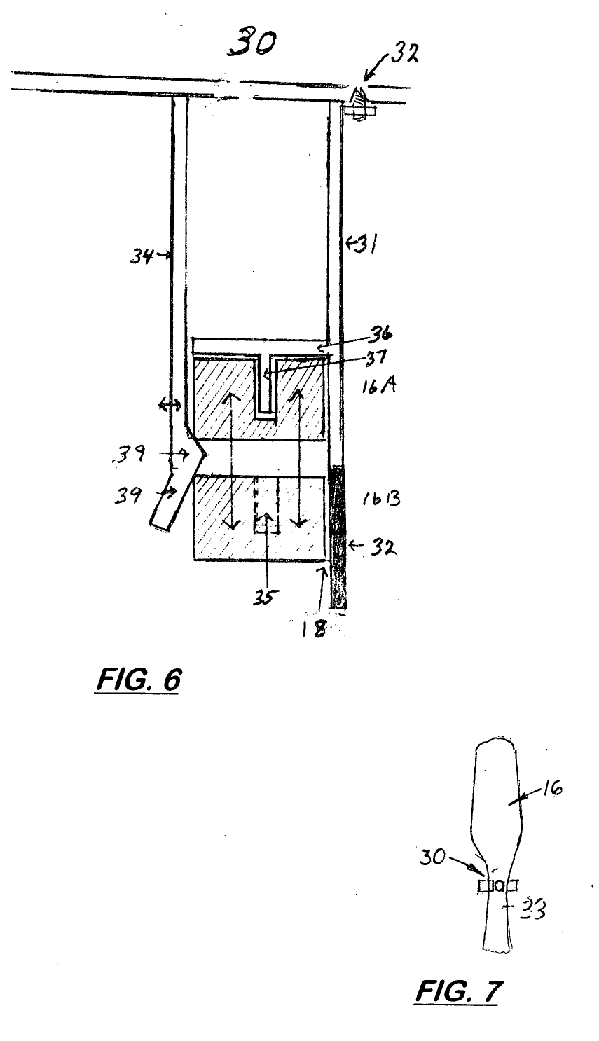

As stated above, each toothbrush 10 is attached to the inside surface 61 of the door 60 with spring clamp retainer 30. In the first embodiment shown in FIGS. 1-3, there are seven spring clamp retainers 30 that securely hold seven, parallel toothbrushes 10 in a longitudinally aligned position in the inside surface 61 of the door 60. FIG. 6 is a detailed top plan view of the spring clamp retainers 30 that allow easy insertion and extraction of the toothbrushes 10 and slight rotational adjustment. As shown in FIGS. 6 and 7, each spring clamp retainer 30 includes a fixed arm 31 perpendicular aligned and extending inward from the inside surface 61 of the door 60. A screw 32 is used to securely attach the fixed arm 31 to the inside surface 61. Attached to the distal end of the fixed arm 31 is a short cross member 32. Attached to the cross member 32 is an inward extending peg 37. Formed on each toothbrush head is a small hole 11 designed to receive the peg 37.

Aligned parallel to the fixed arm 31 is a spring arm 34. Attached to the distal end of the spring arm 34 is a diagonally aligned latch 39 that extends over the outer edge of the toothbrush 10 when the toothbrush head 16 is properly position on the retainer 30 and attached to the peg 37 to hold the toothbrush head 16 on the spring clamp retainer 30.

Located below each spring clamp receiver 30 and also attached to the inside surface 61 of the door 60 is a U-shaped brush holder 38. During use, the brush holder 38 receives the flat distal end of the toothbrush 10. After seating the bottom end of the toothbrush 10 into the brush holder 38, the side of the neck of the toothbrush 10 is placed against the extension 32 on the fixed arm 31. The other side of the neck brushes against the sloping edge of the latch 39 on the spring arm 34. Once placed in this position, the toothbrush 10 is then pressed inward so that the peg 37 is inserted into the hole 37. The toothbrush head is then in proper position against the stop 36.

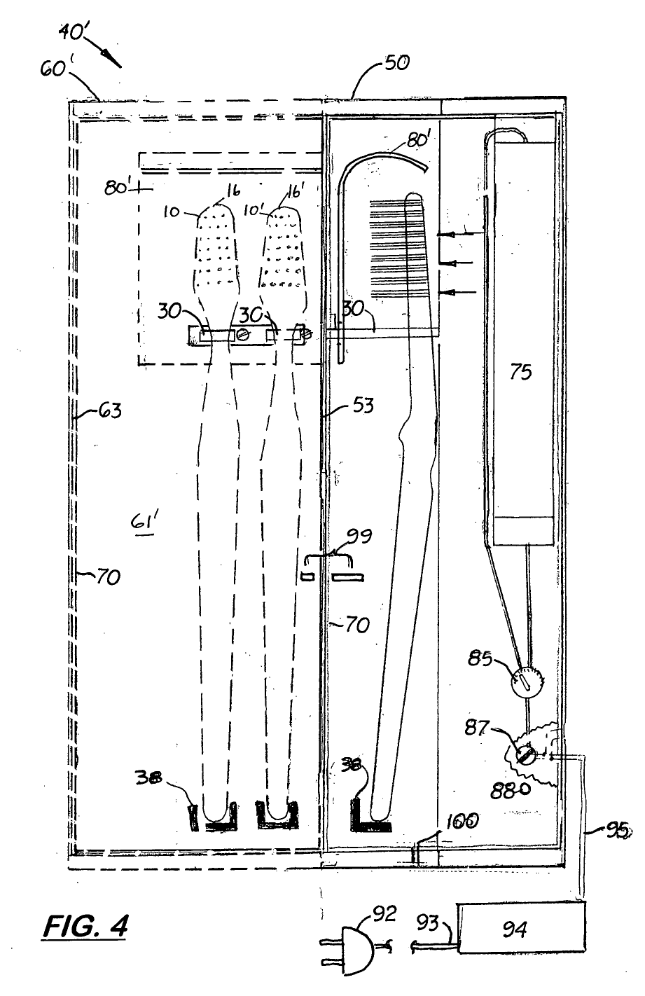

In a second embodiment, shown in FIG. 4, the cleaner, denoted 40′, is a narrow rectangular box structure designed to hold two toothbrushes 10, 10′ on the inside surface 61′ of the door 60′. An elongated germicidal light source 75 is longitudinally aligned in the main body 60′ directly opposite the toothbrush heads 16, 16′. A short concave reflector 80′ is mounted on the door 60′ and directly behind the toothbrush heads 16, 16′.

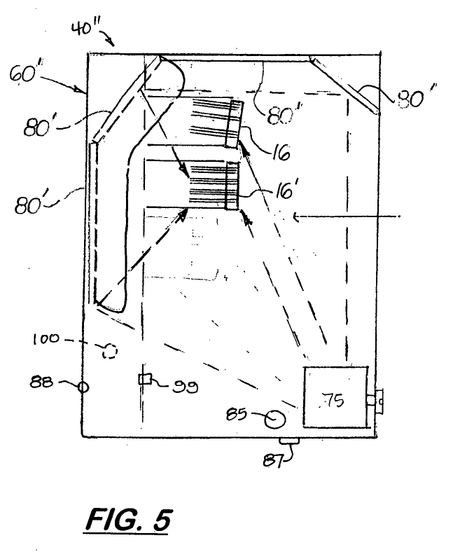

FIG. 5 is top plan view of the second embodiment of the invention shown in FIG. 4 with the light source 75 more clearly shown being longitudinally aligned and mounted in the corner of the main body 50. A plurality of the reflectors 80 are disposed on the opposite corners and on the sidewall that are used to direct light from the germicidal light source to the bristles on the heads.

On both embodiments, a timer 85 is mounted on the outside of the main body and used to control the length of time the light source 75 is lit. Typically, about 60 seconds is all that is needed to completely sterilize the heads 16 and bristles 20. However, if the toothbrushes are wet when placed into the cleaner, additional time may be needed. The timer 85 automatically turns off the light source at the set time on the timer 85. Also located on the outside surface of the main body 50 is an on/off switch 87 used to manually control activation of the light source 75.

Also with each embodiment, a light peep hole 88 is provided on the side of the main body 50 that enables the user to determine if the light source 75 is working and not burned out. The peep hole 88 is covered with suitable material to prevent any germicidal light from escaping.

An electrical plug 92 and wires 93, 95 are connected to a 24 volt transformer 94 to provide electrical power to the light source 75.

There is also a cutoff switch 99 that prevents the light source 75 from being activated if the door 60 is open or ajar and not sealed. The switch 99 is an additional safety measure. An optional drain hole 100 is provided on the bottom of the main body 50 to allow water from the toothbrushes 10 to escape. The drain hole 100 is located on the main body 50 in such a way that ultraviolet light does not escape and cause injury.

As mentioned above, the position of the head 16 of the toothbrush 10 in relation to the germicidal light source 75 is critical. During use, the location of the light source 75 must be positioned in the main body 50, 50′ so that light rays from the light source 75 approaches the ends of the bristles 20 at, or greater, than the critical angle in order to achieve total internal reflection in the bristles. This is made possible by adjusting the toothbrushes by means of the screw 32 on the fixed arm or the spring clamp retainer 30 and my adjusting the position of the light source 75 using the screw 78. Typically, the screws 78 are set and fixed in position during mass production.

In compliance with the statute, the invention described herein has been described in language more or less specific as to structural features. It should be understood, however, that the invention is not limited to the specific features shown, since the means and construction shown is comprised only of the preferred embodiments for putting the invention into effect. The invention is therefore claimed in any of its forms or modifications within the legitimate and valid scope of the amended claims, appropriately interpreted in accordance with the doctrine of equivalents.

1. A germicidal toothbrush, comprising:
a. a head;
b. a handle, and,
c. a plurality of bristles perpendicular aligned and extending through on said handle, each said bristles having exposed ends and made of a fiber optic capable of transmitting germicidal light.

2. A germicidal toothbrush cleaner, comprising:
a. a main body;
b. a door attached to said main body;
c. a germicidal light source located inside said main body;
d. an On/Off switch connected to said germicidal light source;
e. a reflector; and,
f. at least one toothbrush holder used to hold a toothbrush at a set position inside said main body so that light rays produced from said germicidal light source are transmitted directly onto or reflected off said reflector and then onto the exposed ends of the bristles on a toothbrush attached thereto at a critical angle.

3. The cleaner, as recited in claim 2, wherein said holder allows a toothbrush attached thereto to be adjusted in position with respect to said light source.

4. The cleaner, as recited in claim 2, wherein said germicidal light source is a florescent lamp.

5. The cleaner, as recited in claim 2, wherein said reflector is located around the head of a toothbrush attached to said toothbrush holder.

6. The cleaner, as recited in claim 2, wherein said germicidal light source is longitudinally aligned in said main body opposite said tooth brush holder.

7. The cleaner, as recited in claim 6, wherein said germicidal light source is located in the corner of said main body opposite said reflector.

8. The cleaner, as recited in claim 2, further including a drain hole formed on said main body to allow water to drain from said main body.

9. The cleaner, as recited in claim 2, further including means for adjusting the position of said lamp in said main body.

10. The cleaner, as recited in claim 2, further including means for adjusting the position of said toothbrush holder in said body member.

11. The cleaner, as recited in claim 2, further including means to determine if said lamp is activated when said door is closed.

12. The cleaner, as recited in claim 2, wherein said toothbrush holder automatically aligns a toothbrush inside said main body relative to said light source.

13. Method of sterilizing a toothbrush, comprising the following steps:
a. selecting a cleaner with a germicidal light source located therein;
b. selecting a toothbrush with a plurality of bristles attached thereto, said bristles having opposite exposed ends and being made of fiber optic;
c. placing said toothbrush inside said cleaner so that light from said germicidal light source is transmitted at or near the critical angle of reflection on said exposed ends of said bristles;
d. activating said germicidal light so that said toothbrush bristle are sterilized by said germicidal light source.
